(19) United States
(12) Patent Application Publication
(10) Pub. No.: US 2003/0190710 A1
(43) Pub. Date: Oct. 9, 2003

(54) Control of glycoforms in IgG

(76) Inventors: Ruth L. deVries, King of Prussia, PA (US); Thomas Bruce Vickroy, King of Prussia, PA (US)

Correspondence Address: GLAXOSMITHKLINE, Corporate Intellectual Property - UW2220, P.O. Box 1539, King of Prussia, PA, 19406-0939, US

(21) Appl. No.: 10/109,117

(22) Filed: Mar. 28, 2002

(51) Int. Cl. C12P 21/04

(52) U.S. Cl. 435/70.21

(57) ABSTRACT

The present invention provides a method to control the level of non-glycosylated heavy chain variant of an IgG in a CHO cell culture process by adjusting temperature and batched medium osmolality.

This application claims the benefit of U.S. Provisional Application No. 60/279,026, filed Mar. 27, 2001.

### FIELD OF THE INVENTION

This invention relates to processes for controlling glycoforms in recombinantly produced IgG.

### BACKGROUND OF THE INVENTION

Monoclonal antibodies (IgG isotypes) are produced using a variety of expression systems. The majority of the expression systems used for production of monoclonal antibodies are mammalian and include host systems such as chinese hamster ovary (CHO), hybridoma and myeloma cells or their derivatives.

Antibodies significantly differ from other recombinant proteins in their glycosylation patterns. Glycosylation tends to be highly conserved in IgG molecules with a single N-linked biantennary structure at Asn297, which is buried between the CH2 domains, forming extensive contacts with the amino acid residues within CH2. Typically, in recombinantly produced IgG, there is heterogeneous processing of the core oligosaccharide structures attached at the Asn297 site and the IgGs exist in multiple glycoforms. In contrast, non-IgG proteins produced in CHO cells may have multiple N-linked glycosylation sites. Examples are interferon-γ (IFN-γ) which has two glycosylation sites at Asn25 and Asn97 and human tissue plasminogen activator (t-PA) which has 3 sites at Asn117, Asn184 and Asn448.

IgG N-linked glycoforms can vary by site occupancy of the Asn site, i.e., macroheterogeneity, or by variation in the oligosaccharide structure at the glycosylation site, i.e., microheterogeneity. This includes variations in sugar residues, in the length of the oligosaccharide or in the number of branches making up the oligosaccharide (biantennary and triantennary structures). See Jenkins, Parekh, James, (1996) Nature Biotechnology, 14:975-981.

More specifically, in CHO cell processes, there are three major variations observed in the glycoforms produced, namely, terminal oligosaccharide form in the structure, length of the structures and non-glycosylated heavy chain (NGHC). In CHO lines, NGHC formation can be linked to glucose depletion as has been demonstrated in other cell culture systems. See Stark, Heath, (1979) Archives of Biochemistry and Biophysics, 192 (2):599-609, Turco, (1980) Archives of Biochemistry and Biophysics, 205 (2):330-339 and Davidson, Hunt, (1985) Journal of General Virology, 66:1457-1468.

Several investigators have observed glycoform control in IgG preparations under conditions different than those described in the present invention. For example, galactosylation was reduced in IgG produced by a murine B-lymphocyte hybridoma cell line cultured at low dissolved oxygen. See Kunkel, Jan, Butler, Jamieson, (2002) Biotechnol. Prog., 16:462-470. In another IgG-producing hybridoma line, the method of culturing, either in ascites, serum-free or serum-supplemented medium, resulted in different patterns of glycosylation. See Patel, Parekh, Moellering, (1992) Biochem. J., 285:839-845.

U.S. Pat. No. 5,705,364 describes a process for controlling sialic acid content in a glycoprotein produced in a mammalian host cell such as CHO by controlling temperature and osmolality in the presence of an alkanoic acid.

In non-IgG proteins, culture pH and ammonia concentration affected glycosylation of recombinant mouse placental lactogen proteins produced in CHO cells. See Borys, Linzer, Papoutsakis, (1993) Bio/Technology, 11:720-724 and Borys, Linzer, Papoutsakis, (1993) Biotechnology and Bioengineering, 43:505-514. In CHO-produced human follicle stimulating hormone, an increase in specific productivity by varying the steady state of dissolved oxygen plus the addition of butyrate corresponded to an increase in sialic acid content. See Chotigeat, Watanapokasin, Mahler, Gray, (1994) Cytotechnology, 15:217-221. Glycosylation of an artificial N-glycosylation recognition site in recombinant hu-IL-2 produced by BHK-2 was affected by ammonia and glucosamine. Major differences were observed in sialylation, proximal fucosylation and antennarity. See Gawlitzek, Valley, Nimtz, Wagenr, Conradt, Animal Cell Technology: Developments towards the 21st Century, 379-384. Site occupancy in CHO-produced tissue plasminogen activator increased over the length of batch culture and was also affected by butyrate and temperature. See Anderson, Bridges, Gawlitzek, Hoy, (2000) Biotechnology and Bioengineering, 70:25-31.

Glycosylation of IFN-γ produced in CHO has been shown to be sensitive to multiple culture factors. In batch culture, the non-glycosylated form increased from 3-5% at 3 hours up to 30% of total IFN-γ at 195 hours. See Curling, Hayter, Baines, Bull, Gull, Strange, Jenkins, (1990) Biochem. J. 272:333-337. Prolonged culture was also shown to increase oligomannose and truncated structures at Asn 97. See Hooker, Goldman, Markham, James, Ison, Bull, Strange, Slamon, Baines, Jenkins, (1995) Biotechnology and Bioengineering, 48:639-648. The lipid supplement, ExCyte, has also been shown to impact the proportion of fully glycosylated IFN-γ in culture. Also, partially substituting bovine serum albumin (BSA) in the medium with a fatty acid free BSA improved glycosylation. See Jenkins, Castro, Menon, Ison, Bull, (1994) Cytotechnology, 15:209-215. It has also been suggested that Lutrol F68 may affect IFN-γ glycosylation. See Castro, Ison, Hayer, Bull, (1995) Biotechnol. Appl. Biochem. 21:87-100. Primatone RL, an animal tissue hydrolysate, affects sialylation of IFN-γ in batch and fed-batch mode. See Gu, Zie, Harmon, Wang, (1997) Biotechnology and Bioengineering, 56:353-360. Glucose limitation is suggested to affect site occupancy of IFN-γ due to a reduction in nucleotide biosynthesis. See Nyberg, Balcarcel, Follstad, Stephanopoulos, Wang, (1998) Biotechnology and Bioengineering, 62:336-347.

The contribution of the oligosaccharide side chain to IgG function has been greatly debated. One of the key functions of the carbohydrate structure is in the complement fixation pathway and the extent of glycosylation has been directly correlated to antibody-dependent cellular cytotoxicity (ADCC) and recruitment of complement. See Jeffries, Lund, Pound, (1998) Immunology Review, 163:50-76. Fc-receptor binding has been reduced in chimeric mouse-human IgG that are non-glycosylated. See Tao, Morrison, (1989) The Journal of Immunology, 143:2595-2601. Carbohydrate structures also have an effect on protein folding, oligomer assembly and secretion, and in vivo clearance of the glycoprotein. Some glycoforms can be antigenic, prompting regulatory agencies to require increased analysis of the carbohydrate structures in recombinant proteins. See Jenkins, Parekh, James, (1996) Nature Biotechnology, 14:975-981 Further, governmental regulatory requirements for therapeutic IgGs mandate consistency in product preparations. Thus, a need exists for processes that minimize the production of multiple glycoforms of IgG proteins.

### SUMMARY OF THE INVENTION

One aspect of the present invention is a method for controlling the level of an IgG NGHC in cell culture comprising adjusting the culture temperature of the culture.

Another aspect of the present invention is a method for controlling the level of an IgG NGHC in cell culture comprising adjusting the batched medium osmolality of the culture.

Another aspect of the present invention is a method for controlling the level of an IgG NGHC in cell culture comprising adjusting the culture temperature and the batched medium osmolality of the culture.

### BRIEF DESCRIPTION OF THE DRAWINGS

FIG. 1 is a graph of experimental results demonstrating the effect of culture temperature on NGHC formation.

FIG. 2 is a graph of experimental results demonstrating the effect of culture temperature and batched medium osmolality on NGHC formation.

FIG. 3 is a graph of a statistical regression of experimental results predicting the effect of culture temperature and batched medium osmolality on NGHC formation.

FIG. 4 is a graph of experimental results demonstrating the reduction of NGHC in two 1200 L pilot plant batches by increasing culture temperature and reducing batched medium osmolality.

FIG. 5 is a graph of experimental results demonstrating the reduction of NGHC using an in-house and two commercially available media by manipulating temperature and batched medium osmolality.

### DETAILED DESCRIPTION OF THE INVENTION

All publications, including but not limited to patents and patent applications, cited in this specification are herein incorporated by reference as though fully set forth.

The present invention provides a method to control the level of IgG non-glycosylated heavy chain (NGHC) in a cell culture process producing a recombinant monoclonal antibody. By adjustment of the culture temperature, adjustment of the batched medium osmolality or adjustment of both temperature and batched medium osmolality of a cell culture such as a production culture, the level of NGHC can be decreased or increased. Batched medium osmolality of a final seed culture used to inoculate a production culture can optionally be adjusted to match that of the production culture.

In one embodiment of the invention, NGHC can be decreased by increasing the temperature of the cell culture. Likewise, NGHC can be increased by decreasing the temperature of the cell culture. Preferred temperatures for the process are within a range of about 33° C. to about 35° C.

In another embodiment of the invention, NGHC can be decreased by decreasing the batched medium osmolality in the cell culture. Likewise, NGHC can be increased by increasing the batched medium osmolality of the cell culture. Preferred osmolalities for the process are within a range of about 285 mOsm to about 417 mOsm.

In another embodiment of the invention, NGHC level can be decreased by combining increased temperature and low batched medium osmolality of the cell culture.

In yet another embodiment of the invention, NGHC level can be increased by combining decreased temperature and increased batched medium osmolality of the cell culture.

The process of the invention provides for NGHC control at a constant level at any temperature within the preferred range if batched medium osmolality is constant. Likewise, NGHC can be controlled at a constant level at any batched medium osmolality within the preferred range if temperature is held constant.

The present invention will now be described with reference to the following specific, non-limiting Examples.

### EXAMPLES

Media Formulations

For Examples 1 through 6, media compositions consisted of a basal formulation containing amino acids, salts, trace elements, and vitamins similar to those described in PCT International Publication No. WO 92/05246. Yeast hydrolysate such as TC yeastolate was added in an amount of 5 g/L or 10 g/L. Supplemental glucose was added at either 4.5 g/L or 9 g/L. The medium was supplemented with ferric fructose or ferric EDTA, recombinant insulin and a lipid mixture. Sodium bicarbonate was added to batched media as a buffer and methotrexate was added to batched media as a selective agent to maintain expression of the recombinant protein. The surfactant Lutrol F68 was also added to batched media. Batched medium osmolality for seed and production bioreactors are stated in each Example. Osmolality of the batched medium was adjusted by adding NaCl and KCl. During cultivation in bioreactors, sodium carbonate was added as needed for pH control. Media was sterilized by either 0.1 or 0.2 micron filtration.

In Example 7, proprietary medium as described above was used. Additionally, cells were adapted to two commercially available media, CD-CHO (Invitrogen, Rockville, Md.) and EX-CELL 325 (JRH Biosciences, Lenexa, Kans.). All cultures were then evaluated for NGHC production at various temperatures and batched medium osmolalities.

Scale-Up and Production Conditions for Examples 1 through 5

Data was generated by cultivation of a recombinant CHO cell line producing an IgG1 anti-CD4 monoclonal antibody in shake flasks and 3 L Applikon bioreactors. This antibody is described as CE9.1 in U.S. Pat. No. 6,136,310. Shake flask scale-up cultures were grown in medium with an osmolality in the range of 370-380 mOsm and shaken at 150 RPM in a 5% carbon dioxide incubator at 37° C. Cells were passed on a 3-4 day schedule with a target seeding density of 600,000 viable cells/mL. On passage days, cells were trypsinized and counted using trypan blue exclusion and a hemacytometer for percent viability and a ZM Coulter Counter for total cell count.

3L seed bioreactors were operated at 37° C. at 200 RPM with 5 mL/min O2 sparge on demand to maintain dissolved oxygen at setpoint, 5 mL/min 100% carbon dioxide to headspace on demand or 1.5M Na2CO3 on demand to control pH in the setpoint band of 6.9-7.0. Bioreactors had a constant 20 mL/min air overlay.

3L production bioreactors were operated the same as seed reactors except for temperature and pH setpoints which varied depending on the example. Every one or two days, cells in seed and production bioreactors were trypsinized and counted using trypan blue exclusion and a hemacytometer for percent viability and a ZM Coulter Counter for total cell count. Batched media osmolalities were as specified in each example.

Scale-Up and Production conditions for Example 6

Shake flask scale-up cultures of the recombinant cell line were run as described above. Cells were then scaled to an 80 L seed bioreactor with a 17 L culture volume. After several days of cultivation at 37° C. and pH 6.9-7.0 with dissolved oxygen of 48 mm Hg, the bioreactor was fed fresh medium up to 71 L. After several days of cultivation at 37° C. and pH 6.9-7.0 with dissolved oxygen of 48 mm Hg, the 71 L culture was used to inoculate a 750 L ABEC seed bioreactor at a 300 L culture volume. After several days of cultivation at 37° C. and pH 6.9-7.0 with dissolved oxygen of 48 mm Hg, the 300 L was used to inoculate a 1500 L ABEC production bioreactor with a 1200 L culture volume. The production reactor was operated at temperatures specified in the example and pH 6.9-7.0 with dissolved oxygen of 48 mm Hg. Batched medium osmolality for the 80 L seed reactor was 370-380 mOsm. Batched medium osmolality for the 750 L ABEC seed reactor and the 1500 L ABEC production reactor was as specified in the Example.

Scale-Up and Production Conditions for Example 7

Shake flask scale-up cultures of the recombinant cell line were shaken at 150 RPM, in a 5% carbon dioxide incubator at 37° C. Cells were passed on a 3-4 day schedule with a target seeding density of 800,000 VCC/mL. On passage days, cells were trypsinized and counted using trypan blue exclusion and a hemacytometer for percent viability and a Z1 Coulter Counter for total cell count.

A vial of cells was thawed and scaled using medium as described above for 4 passages. A subset of these cells continued to be scaled using this medium and two other portions were adapted to two commercial media, CD-CHO (Invitrogen) and EXCELL-325 (JRH Biosciences), using the following ratios of in-house media to vendor media over 5 passages: 100/0, 50/50, 0/100. Batched medium osmolalities for scale-up and adaptation were 358 mOsm for in-house medium and 353 mOsm for the commercial media. For the last seed passage at 37 C., cells in each medium were subcultured into like media batched at low, mid, and high osmolalities. For in-house medium, these were 310, 358 and 405 mOsm respectively. For the commercial media, these were 300, 353 and 405 mOsm respectively. At the end of this final seed passage, flasks at each medium/osmolality treatment were used to inoculate production shake flasks with like medium/osmolality treatment. Production flasks for each medium/osmolality treatment were then incubated at 33° C., 34° C. or 35° C. Production cultures were harvested when viabilities were approximately 60% or lower. This occurred after approximately 14-17 days of cultivation. Flasks were also monitored for residual glucose to ensure it was not depleted on the day of harvest.

Analysis for NGHC Levels

In Examples 1 through 5, cells were filtered from the production cultures. For each batch, product was captured and concentrated on a protein A affinity column. The product eluate was then analyzed for % NGHC by densitometry scans of reduced SDS-PAGE gels. NGHC was reported as percentage of the total heavy chain or as percentage of total heavy and light chain as specified in each example.

In Example 6, cells were filtered from the production cultures. For each batch, product underwent capture and concentration on an affinity column and then was processed through 2 more chromatography steps. Final product was then analyzed for % NGHC by densitometry scans of SDS-PAGE gels.

In Example 7, cells were filtered from the production cultures. For each batch, product was captured and concentrated on an affinity column. The product eluate was then analyzed for % NGHC by a capillary SDS-PAGE separation performed on a micro-capillary array using an Agilant Bioanalyzer.

### EXAMPLE 1

Effect of Culture Temperature on NGHC Formation

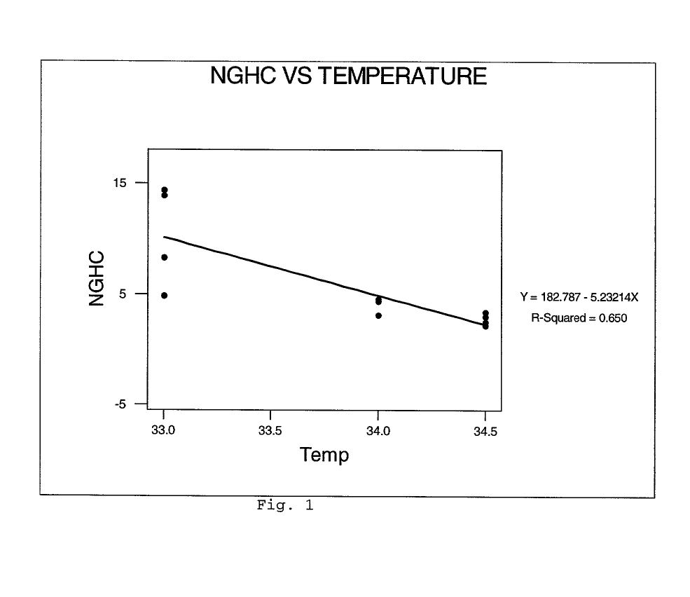

The impact of culture temperature on NGHC formation in culture was first observed in 3L Applikon bioreactors with a 2L culture volume. Standard process setpoints for the production reactor were temperature of 34° C., pH of 6.9-7.0 and dissolved oxygen of 30%. A factorial study was designed to evaluate the impact of seed culture quality, culture temperature, pH, and dissolved oxygen on titer. Seed culture quality was varied by changing the inoculation density and the age of the inoculum into the seed reactor from those used in a typical seed reactor. The combinations of factors that were tested and resultant NGHC levels as percent of heavy chain are show in Table 1. Statistical analysis indicated that culture temperature was statistically significant in the formation of NGHC (p<0.002). Adjusted R-sq was 61.5% indicating the temperature explains only a portion of the variation of NGHC. FIG. 1 illustrates the linear regression.

TABLE 1

| Seed | | | DO | |
|---|---|---|---|---|
| Quality | Temp | pH | mm Hg | % NGHC |
| − | 34.5 | 6.8 | 24 | 2.2 |
| − | 34.5 | 7.0 | 112 | 2.5 |
| + | 34.5 | 7.0 | 24 | 3 |
| − | 34 | 6.9 | 48 | 3.1 |
| + | 34.5 | 6.8 | 112 | 3.4 |
| + | 34 | 6.9 | 48 | 4.4 |
| 0 | 34 | 6.9 | 48 | 4.5 |
| (control) | | | | |
| 0 | 34 | 6.9 | 48 | 4.6 |
| (control) | | | | |
| − | 33 | 6.8 | 112 | 4.9 |
| + | 33 | 6.8 | 24 | 8.3 |
| + | 33 | 7.0 | 112 | 13.9 |
| − | 33 | 7.0 | 24 | 14.4 |

### EXAMPLE 2

Effect of Culture Temperature and Batched Medium Osmolality on NGHC Formation

The effect of culture temperature and batched medium osmolality on titer and formation of NGHC was determined. Culture temperatures between 33.4° C. and 34.2° C. and osmolalities of 340 mOsm, 370 mOsm, and 400 mOsm were evaluated in 3L bioreactors with a 2L culture volume. NGHC as percent of heavy chain is shown in Table 2. The data confirms the effect of culture temperature on NGHC formation as described in Example 1 and also suggests that higher batched medium osmolality contributes to NGHC formation. IVC represents intergral of viable cells.

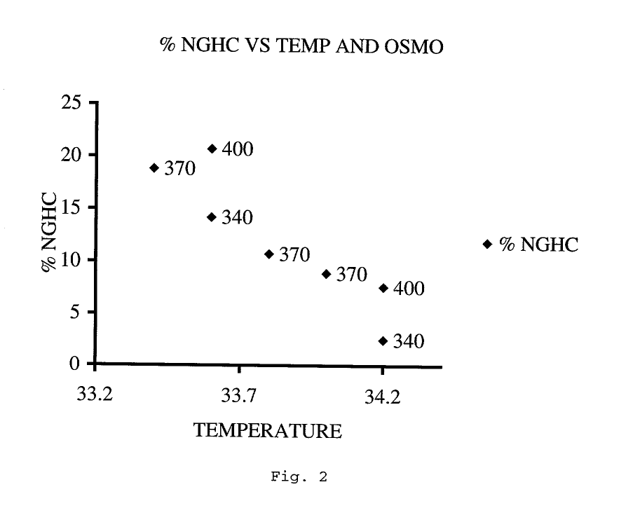

All cultures ended with residual glucose indicating that increased NGHC levels did not correlate with glucose depletion. FIG. 2 illustrates NGHC versus temperature and osmolality.

TABLE 2

| | | | RESIDUAL | |
|---|---|---|---|---|
| TEMP | OSMO | IVC | GLUCOSE g/L | % NGHC |
| 33.6 | 400 | 16.32 | 4.02 | 20.7 |
| 33.4 | 370 | 15.15 | 4.94 | 18.8 |
| 33.6 | 340 | 16.52 | 4.93 | 14.2 |
| 33.8 | 370 | 17.48 | 4.47 | 10.7 |
| 34.0 | 370 | 22.25 | 3.09 | 8.9 |
| 34.2 | 400 | 26.5 | 1.36 | 7.6 |
| 34.2 | 340 | 26.02 | 2.92 | 2.5 |

### EXAMPLE 3

Enrichment and Reduction of NGHC by Manipulating Culture Temperature and Batched Medium Osmolality

Four 3L bioreactors with 2L culture volume were run at varying temperatures (33°, 33.2°, 35° and 34.4° C.) and batched medium osmolalities (340, 370 and 400 mOsm) in an attempt to enrich NGHC and minimize NGHC based on data generated in Example 1 and Example 2. NGHC levels as percent of heavy chain are shown in Table 3.

TABLE 3

| | | | RESIDUAL | |
|---|---|---|---|---|
| TEMP | OSMO | PURPOSE | GLUCOSE g/L | % NGHC |
| 33.0 C. | 370 mOsm | Enrich NGHC | 5.14 | 17.3% |
| 33.2 C. | 400 mOsm | Enrich NGHC | 4.57 | 22.7% |
| 35.0 C. | 340 mOsm | Reduce NGHC | 0 | 3.1% |
| 34.4 C. | 340 mOsm | Reduce NGHC | 1.7 | 2.6% |

EXAMPLE 4

Confirmation of Conditions Resulting in Low NGHC

Eight 3L bioreactors with 2L culture volume were run at temperatures between 34.2° C. and 35.0° C. and batched medium osmolalities of 285 mOsm, 304 mOsm or 331 mOsm. NGHC was less than 3.5% for all batches. NGHC as percent of total protein is shown in Table 4.

TABLE 4

| TEMP | OSMO | % NGHC |
|---|---|---|
| 34.2 | 285 | 2.2 |
| 34.6 | 285 | 2.0 |
| 35.0 | 285 | 2.2 |
| 34.2 | 304 | 2.3 |
| 35.0 | 304 | 2.1 |
| 34.2 | 331 | 3.5 |
| 34.6 | 331 | 2.0 |
| 35.0 | 331 | 2.6 |

### EXAMPLE 5

Further Evaluation of Culture Temperature and Batched Medium Osmolality Effect on NGHC Formation

Twenty-one 2L bioreactors were run varying culture temperatures at 0.2° C. increments between 33.6° and 34.4° and six different batched medium osmolalities between 337 mOsm and 417 mOsm. NGHC as percent of total protein is shown in Table 5. The data indicates that glucose depletion on next to the last day or last day of culture did not correlate with higher NGHC levels.

TABLE 5

| | | RESIDUAL | |
|---|---|---|---|
| TEMP | OSMO | GLUCOSE g/L* | % NGHC |
| 33.6 | 337 | 4.34 | 3.9 |
| 34.2 | 337 | 2.80 | 3.1 |
| 34.4 | 337 | 2.96 | 3.2 |
| 33.6 | 361 | 3.64 | 8.0 |
| 34 | 361 | 1.92 | 3.6 |
| 33.6 | 383 | 3.30 | 7.8 |
| 33.8 | 383 | 2.52 | 8.5 |
| 34 | 383 | 3.01 | 4.8 |
| 34.4 | 395 | 0.91 | 4.0 |
| 33.6 | 417 | 2.55 | 14.2 |
| 34.2 | 417 | 1.07 | 4.7 |
| 34.4 | 417 | 0.63 | 5.0 |
| 33.6 | 337 | 3.97 | 2.5 |
| 33.8 | 337 | 4.11 | 3.6 |
| 34.4 | 337 | 2.15 | 2.3 |
| 34.4 | 355 | 0.23 | 2.7 |
| 34.2 | 383 | 2.18 | 4.8 |
| 33.6 | 395 | 3.51 | 9.9 |
| 34 | 395 | 2.33 | 7.7 |
| 34 | 417 | 2.66 | 8.4 |
| 34.4 | 417 | 0.59 | 2.8 |
| **34.0 | 380 | 2.73 | 5.1 |

*Glucose level on next to last day or last day of production culture
**Internal control for standard process conditions

The data from Example 4 were combined with data from Example 5 for statistical analysis. This analysis produced the following regression equation:

NGHC=3.13−2.57*(TEMP)+1.89*(OSMO)−4.8(TEMP*OSMO)

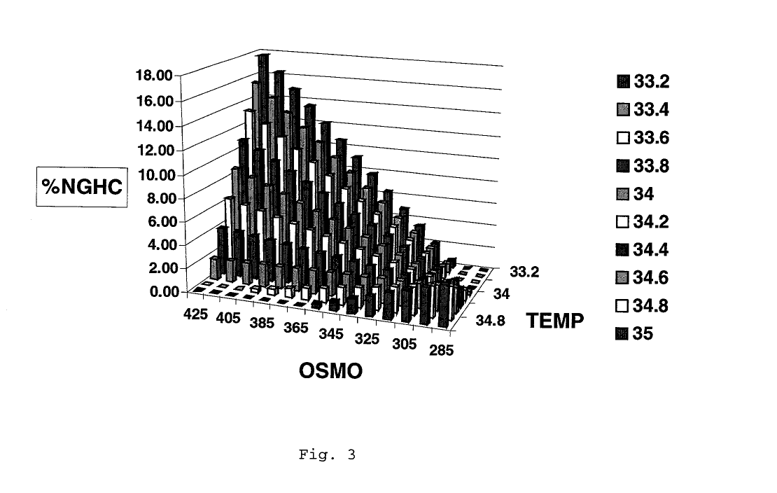

p<0.001 for culture temperature, batched medium osmolality and the interaction of culture temperature and batched medium osmolality. The adjusted R−sq=87.8% and the overall p=0.000. The model is shown graphically in FIG. 3 and predicts that at a temperature between 34.6° C. and 34.7° C., NGHC can be maintained at a constant level. Likewise, at a batched medium osmolality of 316 mOsm, NGHC can be maintained at a constant level for all temperatures between 33.5° C. and 35.1° C.

### EXAMPLE 6

Increasing Culture Temperature and Decreasing Batched Medium Osmolality Reduce NGHC Formation at 1200L Pilot Plant Scale

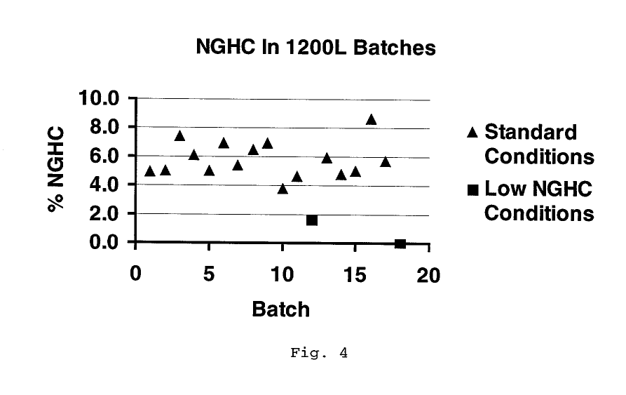

It was demonstrated that NGHC could be reduced in 1200L bioreactor cultures by increasing culture temperature and reducing batched medium osmolality. Standard production bioreactor culture temperature was 33.9° C. and osmolality for seed flasks, seed bioreactors and the production reactor was 370-380 mOsm. In two of 20 batches, the culture temperature of the production bioreactor was increased to 34.5° C. and batched medium osmolality reduced to 305-315 mOsm. For these two batches, the batched osmolality of the final 750L seed bioreactor was also reduced to 305-315 mOsm. Average NGHC for the 18 standard batches was 5.7%. NGHC values as percent of total protein for the 2 “low NGHC” batches were 1.7% and “none detected.” FIG. 4 illustrates NGHC levels as percent of total protein in all batches.

### EXAMPLE 7

Impact of Culture Temperature and Batched Medium Osmolality on NGHC Formation in In-house Medium and Two Commercially Available Cell Culture Media

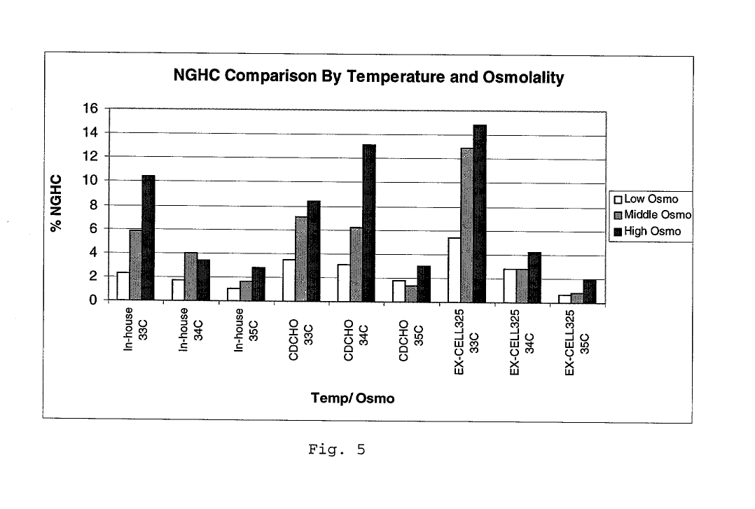

It was demonstrated that NGHC could be reduced using in-house medium and two commercially available media, CD-CHO (Invitrogen) and EXCELL-325 (JRH Biosciences) by adjusting temperature and osmolality. For each medium, three temperatures and three osmolalities were evaluated in shake flasks. Temperatures were 33° C., 34° C., and 35° C. Osmolalities for the proprietary medium were 310, 358, and 405 mOsm. Osmolalities for the commercial medium were 300, 353, and 405 mOsm. Table 6 and FIG. 5 indicate a trend similar to that demonstrated in previous examples. At low temperature, NGHC formation as % of total protein is higher and more sensitive to batched medium osmolality than at mid and high temperatures. Glucose was not depleted in any of the tested conditions.

TABLE 6

| Low Osmolality | | Mid Osmolality | | High Osmolality | |
|---|---|---|---|---|---|
| Media/Temp | % NGHC | Media/Temp | % NGHC | Media/Temp | % NGHC |
| In-house 33 C. | 2.3 | In-house 33 C. | 5.8 | In-house 33 C. | 10.4 |
| In-house 34 C. | 1.7 | In-house 34 C. | 4 | In-house 34 C. | 3.4 |
| In-house 35 C. | 1 | In-house 35 C. | 1.6 | In-house 35 C. | 2.8 |
| CDCHO 33 C. | 3.5 | CDCHO 33 C. | 7.1 | CDCHO 33 C. | 8.4 |
| CDCHO 34 C. | 3.1 | CDCHO 34 C. | 6.2 | CDCHO 34 C. | 13.1 |
| CDCHO 35 C. | 1.8 | CDCHO 35 C. | 1.3 | CDCHO 35 C. | 3 |
| EX-CELL325 33 C. | 5.4 | EX-CELL325 33 C. | 12.9 | EX-CELL325 33 C. | 14.8 |
| EX-CELL325 34 C. | 2.8 | EX-CELL325 34 C. | 2.8 | EX-CELL325 34 C. | 4.2 |
| EX-CELL325 35 C. | 0.68 | EX-CELL325 35 C. | 0.82 | EX-CELL325 35 C. | 1.9 |

The present invention may be embodied in other specific forms without departing from the spirit or essential attributes thereof, and accordingly, reference should be made to the appended claims, rather than to the foregoing specification, as indicating the scope of the invention.

1. A method for controlling the level of an IgG non-glycosylate heavy chain (NGHC) produced in mammalian cell culture comprising adjusting the temperature of the culture.

2. A method for controlling the level of an IgG NGHC produced in mammalian cell culture comprising adjusting the batched medium osmolality of the culture.

3. A method for controlling the level of an IgG NGHC produced in mammalian cell culture comprising adjusting the temperature and the batched medium osmolality of the culture.

4. The method of claim 1, 2 or 3 wherein the cell culture host cell is a chinese hamster ovary (CHO) cell.

5. The method of claim 4 wherein the cell culture is a production culture.

6. The method of claim 4 wherein the level of NGHC is decreased by decreasing batched medium osmolality and increasing temperature.

7. The method of claim 4 wherein the level of NGHC is decreased by decreasing batched medium osmolality.

8. The method of claim 4 wherein the level of NGHC is increased by increasing batched medium osmolality.

9. The method of claim 4 wherein the level of NGHC is increased by decreasing temperature and increasing batched medium osmolality.

10. The method of claim 4 wherein the level of NGHC is decreased by increasing temperature.

11. The method of claim 4 wherein the level of NGHC is increased by decreasing temperature.

12. The method of claim 4 wherein the temperature of the culture is adjusted to about 33° C. to about 35° C.

13. The method of claim 4 wherein the osmolality is adjusted to about 285 mOsm to about 417 mOsm.

14. The method of claim 4 wherein the temperature of the culture is adjusted to about 33° C. to about 35° C. and the osmolality is adjusted to about 285 mOsm to about 417 mOsm.
